(19) United States
(12) Patent Application Publication
(10) Pub. No.: US 2022/0000428 A1
(43) Pub. Date: Jan. 6, 2022

(54) METHOD FOR DETERMINING A PREDICTION MODEL, METHOD FOR PREDICTING THE EVOLUTION OF A K-UPLET OF MK MARKERS AND ASSOCIATED DEVICE

(71) Applicant: QYNAPSE, PARIS (FR)

(72) Inventors: Clarisse LONGO DOS SANTOS, Vanves (FR); Jean-Baptiste MARTINI, Malakoff (FR); Urielle THOPRAKARN, Antony (FR); Bruno VEGREVILLE, Paris (FR)

(21) Appl. No.: 17/293,783

(22) PCT Filed: Nov. 14, 2018
(86) PCT No.: PCT/EP2018/081267

§ 371 Date: May 13, 2021

(51) Int. Cl. A61B 5/00; G06T 7/00; A61B 5/055; G01R 33/48; G16H 50/20

(52) U.S. Cl. CPC A61B 5/7275; G06T 7/0012; A61B 5/0042; A61B 5/055; A61B 5/407; G01R 33/4806; G06T 2207/20076; G06T 2207/10088; G06T 2207/30096; G06T 2207/30016; G06T 2207/30204; G06T 2207/30168; G16H 50/20

(57) ABSTRACT

A method for determining a prediction model for predicting, from an N-uplet of markers Mn, the value of a K-uplet of markers Mk to assist the prognosis of central nervous system pathologies, the method including for each subject of a plurality of subjects, a step of acquiring, at a time TO, an N-uplet of markers Mn, to obtain a plurality of N-uplets of markers Mn; for each subject of the plurality of subjects, a step of acquiring, at a time T* greater than or equal to TO, a K-uplet of markers Mk, to obtain a plurality of K-uplets of marker Mk; and a step of determining, from the plurality of N-uplets of markers Mn and the plurality of K-uplets of markers Mk, a prediction model for associating with any N-uplet of markers Mn acquired at a time T, a K-uplet of marker Mk at a time T+ΔT with ΔT=T*−TO.

### TECHNICAL FIELD OF THE INVENTION

The technical field of the invention relates to the field of disorders of the central nervous system and the aid in predicting the course of these disorders in human subjects. The present invention relates in particular to a method for determining a prediction model of at least one marker for aiding in the prognosis of pathologies of the central nervous system, a method for predicting the course of a marker in a subject for aiding in the prognosis of pathologies of the central nervous system, and the device associated with said methods.

### TECHNOLOGICAL BACKGROUND OF THE INVENTION

Central nervous system diseases affect more than 2 billion people worldwide. Among the neurological conditions, neurodegenerative diseases (e.g. Alzheimer's, Parkinson's) occupy a predominant place due to their severity and their increasing frequency related to the ageing of the population. Worldwide, 50 million people suffer from Alzheimer's disease and 10 million from Parkinson's disease. Inflammatory diseases such as multiple sclerosis in turn affect about 2.3 million people. The ageing of the population in developed countries is accompanied by an increase in memory disorders and related disorders. Epidemiological studies have highlighted the wide variety of dysfunctions that exist in this area and the corresponding symptoms.

The first problem is that of differential diagnosis. Among neurodegenerative diseases, Alzheimer's disease has been the subject of numerous studies. However, the symptoms considered, such as memory disorders, difficulties in orienting oneself in space and time, or behavioural disorders, are not specific to Alzheimer's disease. For a long time, it was considered that the diagnosis of Alzheimer's disease could only be confirmed post mortem, with the demonstration of amyloid plaques and tangles of degenerating neurons in the brain, or at an advanced clinical stage of the disease. In Parkinson's disease, one of the imaging tests (SPECT DaTscan) used today to establish a differential diagnosis between essential tremors and degenerative Parkinson's syndromes does not, on its own, make it possible to differentiate idiopathic Parkinson's disease from the other syndromes (progressive supra-nuclear palsy and multisystematic atrophies), nor does it make it possible to differentiate Parkinson's dementia from Lewy body dementia.

Another major issue is the prognosis. For example, for multiple sclerosis, the manual reading of cerebral and spinal cord lesions as it is done today is not very precise, is tedious and does not constitute a sufficient prognostic marker on its own. However, for this disease as for others, for clinical trials, having a precise prognosis at the time of inclusion of patients will make it possible to carry out shorter studies on smaller cohorts, generating time savings and major savings for pharmaceutical laboratories and biotechnology companies.

On the other hand, for routine clinical practice, the challenge of prognosis is to tailor the therapeutic strategy and the management of patients. In particular, a patient whose disease is likely to progress in the short term (two years) versus the medium term (five years) will require adapted management. Furthermore, while the prognosis of cognitive decline and loss of autonomy are major issues for individualised patient care, they are also major issues for planning resources and for organising carers.

It is therefore important to manage patients and to help predict the course of these diseases, especially at the early stage, in order to hope for a better effectiveness of treatments delaying the progression of symptoms; such a prognosis is also useful for carrying out epidemiological studies and improving knowledge of these disorders.

In order to carry out such a prognosis, patent EP 1 491 889 A2 proposes a method for aiding the diagnosis of Alzheimer's disease in which the course of the level of the A3 peptide (x-41) in the cerebrospinal fluid (or CSF) of a patient is measured. However, such a method requires a sample of this fluid and is therefore invasive. Furthermore, it only gives an indication of the patient's condition after a relatively long time between the two measurements and therefore does not allow for early management.

In order to avoid the use of invasive tests, it has been proposed to use magnetic resonance imaging (or MRI) to identify the disease and predict its course. For example, patent application CA 2 565 646 proposes a system for predicting a clinical state using medical image data. More particularly, the authors propose a predictive model for the course of a patient's clinical state based on the collection of brain volume data obtained by methods including MRI, X-ray imaging, scintigraphy, computed tomography (CT), microwave, infrared, portal or optical imaging, fluoroscopy or Positron Emission Tomography (PET). However, the score provided as a result of the described method is an overall score and does not allow independent access to relevant patient information. Furthermore, the clinical state prediction system is a static model and a change over time of this model is not foreseen. Furthermore, a study published in The Lancet (2014, 614-629, Dubois et al) showed that the application of criteria based solely on brain imaging led to the inclusion, among patients with Alzheimer's disease, of many cases that did not have Alzheimer's disease, although they showed signs that could also be observed in Alzheimer's disease. These false positives could especially explain the low efficacy rate recorded in treatment trials.

In order to reduce or even eliminate false positives, Dubois et al propose the use of new markers, adapted according to the presumed stage of Alzheimer's disease. These markers combine a PET scan and the assay of tau and amyloid proteins in cerebrospinal fluid to confirm the existence of Alzheimer's disease. However, the authors point out that these criteria require expensive and/or difficult-to-implement tests, and should therefore probably be reserved for referral centres. Furthermore, they are not sufficient on their own to rule out the existence of other neurodegenerative diseases.

Similarly, a study by Jussi Mattila et al (J Alzheimer's Dis. 2011; 27(1):163-76) highlighted, especially for Alzheimer's disease, the interest of weighting the contribution of the various markers by their relevance. A weighted index can therefore be constructed from these markers, such that it will be representative of a subject's state. By comparison with a normative base containing previously diagnosed subjects, the proposed method can guide the clinician in his or her diagnostic decisions. However, it is again a prediction from an overall index that does not allow the clinician to access detailed information.

There is therefore a need for a method that can be implemented in a simple and reproducible way to help predict, in a subject who may have disorders related to an impairment of the central nervous system, the course of the different markers characteristic of these pathologies. The diagnosis of diseases such as Alzheimer's or related diseases is multidisciplinary, and always requires a complete clinical examination, but it is desirable to have a tool to help with orientation and prognosis based on the prediction of the course of markers, thus making it possible to classify subjects and to pursue other investigations which will conclude that there is a risk of developing a complete clinical syndrome.

It is also desirable to have a data set comprising marker values, as well as their variation over time, associated with neurological states for a plurality of subjects to allow the implementation of the method for predicting the course of these markers. It is also desirable to be able to develop, if necessary, the course prediction models to take modifications of parameters recorded during their implementation into account.

### SUMMARY OF THE INVENTION

The invention offers a solution to the problems discussed above, by making it possible to predict, from a first plurality of markers, the value of a second plurality of markers. For this, it provides a method for obtaining a prediction model and a method using said model.

A first aspect of the invention relates to a method for determining a prediction model, from an N-tuple of markers Mn, of the value of a K-tuple of markers Mk for aiding in the prognosis of pathologies of the central nervous system, said method comprising:
for each subject of a plurality of subjects, a step of acquiring at a time T0 an N-tuple of markers Mn so as to obtain a plurality of N-tuples of markers Mn;
for each subject of the plurality of subjects, a step of acquiring, at a time T* greater than or equal to T0, a K-tuple of markers Mk so as to obtain a plurality of K-tuples of markers Mk;
a step of determining, from the plurality of N-tuples of markers Mn and the plurality of K-tuples of markers Mk, a prediction model making it possible to associate any N-tuple of markers Mn acquired at a time T with a K-tuple of markers Mk at a time T+ΔT with ΔT=T*−T0.

By virtue of the invention, a prediction model is available which can be used subsequently to predict the value of one or more markers in a subject according to the value of reference markers in the same subject.

Further to the characteristics just discussed in the preceding paragraph, the method according to a first aspect of the invention may have one or more of the following additional characteristics, considered individually or in any technically possible combinations.

In one embodiment, at least one of the markers of the N-tuple of markers Mn or the K-tuple of markers Mk is an imaging marker or a biological marker.

In one embodiment, at least one of the markers of the N-tuple of markers Mn is selected from:
an imaging marker indicative of the volumetry of a part of the subject's brain selected from hippocampal volume, whole brain volume, cerebellum volume, volume of subcortical structures, cortical thickness and opening of cortical-cerebral sulci, said marker being derived from a magnetic resonance image of at least one part of the subject's brain;
an imaging marker indicative of lesion load, such as the volume of white matter lesions, said marker being derived from a magnetic resonance image of at least one part of the subject's brain or spinal cord; and
a brain functional imaging marker selected from markers indicative of glucose metabolism, markers indicative of amyloid load, markers indicative of the dopaminergic system and markers indicative of the level of brain oxygenation.

In one embodiment, at least one of the markers of the N-tuple of markers Mn is selected from:
a cognitive marker of the subject; or
a motor marker of the subject; and
a mood marker of the subject;
a demographic marker of the subject;
a marker of the subject's autonomy;
a marker relating to the subject's stage of progress in the disease.

In one embodiment, at least one marker of the N-tuple of markers Mn is a marker indicative of the concentration in the cerebrospinal fluid of at least one protein selected from Tau, P-tau and Abeta42 proteins, the measurement of said concentration being performed in vitro.

In one embodiment, at least one of the markers of the K-tuple of markers Mk is selected from:
an imaging marker indicative of the volumetry of one part of the subject's brain selected from hippocampal volume, whole brain volume, cerebellum volume, volume of subcortical structures, cortical thickness and opening of cortical-cerebral sulci, said marker being derived from a magnetic resonance image of at least one part of the subject's brain;
an imaging marker indicative of lesion load, such as the volume of white matter lesions, said marker being derived from a magnetic resonance image of at least one part of the subject's brain or spinal cord; and
a brain functional imaging marker selected from markers indicative of glucose metabolism, markers indicative of amyloid load, markers indicative of the dopaminergic system and markers indicative of the level of brain oxygenation.

In one embodiment, at least one of the markers of the K-tuple of markers Mk is selected from:
a cognitive marker of the subject; or
a motor marker of the subject; and
a mood marker of the subject; and
a marker of the subject's autonomy;
a marker of the subject's stage of progress in the disease.

In one embodiment, the method comprises, after the step of acquiring at a time T0 an N-tuple of marker Mn, preferably after the step of acquiring at a time T* greater than or equal to T0 a K-tuple of markers Mk, and before the step of determining a prediction model, a step of correcting the marker outliers.

In one embodiment, the step of correcting the outliers comprises:
for each marker Mn of the N-tuple of markers Mn, a substep of determining the number of subjects for which the value of said marker Mn is judged to be an outlier;
for each marker Mn, if the number of subjects for which the value of the marker Mn in question is judged to be an outlier is greater than a threshold number of subjects, a sub-step of deleting the marker Mn in question, said marker not being taken into account during the step of determining a prediction model;
for each marker Mn, if the number of subjects for which the value of the marker Mn in question is judged to be an outlier is less than or equal to the threshold number of subjects, a sub-step of replacing, for the subjects concerned, the value of the marker Mn in question by the value of the quartile closest to said marker Mn;

the value Xᵢ of a marker Mn is judged to be an outlier for a subject if it does not satisfy the following relationship:

Med−3σ_{b}≤Xᵢ≤Med+3σ_{b}

where Med is the median of the values Xᵢ of the marker Mn for the plurality of subjects, σ_{b} the value of a deviation such that σ_{b}=b×DMed with b a predefined coefficient and DMed=median(|X₁−Med|, . . . , |Xₙ− Med|).

In one embodiment, the method comprises, after the step of acquiring at a time T0 an N-tuple of marker Mn and before the step of determining a prediction model, a step of adding a predetermined value for the missing values of markers Mn.

In one embodiment, the step of adding a predetermined value for the missing values of markers Mn comprises:
a substep of determining the three subjects closest to the subject associated with a missing value of marker Mn;
a substep of calculating the mean value of the missing marker Mn for the three closest subjects;
a sub-step of adding the missing value of the marker Mn, said value being equal to the mean value calculated in the sub-step of calculating the mean value of the missing marker Mn.

In one embodiment, the method comprises, after the step of acquiring at a time T0 a N-tuple of markers Mn and before the step of determining a prediction model, a step of controlling the quality of the imaging markers.

In one embodiment, the step of controlling the quality of the imaging markers comprises, for each imaging marker:
a sub-step of checking compliance with the acquisition procedure that enabled determination of the value of the marker, the imaging markers associated with an image that does not comply with the procedure being considered to be missing;
when the image has been obtained in compliance with the procedure, a sub-step of checking the quality of the image that allowed the determination of the value of the marker so as to obtain, for each image, a plurality of descriptors;

the step of controlling the quality of the imaging markers comprising, for each plurality of descriptors:
a sub-step of checking the quality of the plurality of descriptors using a classifier;
when the quality of the plurality of descriptors is above a predetermined threshold, the imaging marker is retained;
when the quality of the plurality of descriptors is below a predetermined threshold, the imaging marker is considered as missing.

In one embodiment, the step of acquiring an N-tuple of markers Mn so as to obtain a plurality of N-tuples of markers Mn is performed for a plurality of times T0, the step of determining a prediction model taking the N-tuples of markers Mn into account for each time T0 of the plurality of times T0.

A second aspect of the invention relates to a method for predicting the course of a K-tuple of markers Mk in a subject for aiding the prognosis of pathologies of the central nervous system using a prediction model of a K-tuple of markers Mk obtained using a method according to one of the preceding claims, characterised in that it comprises:
a step of acquiring at a time T an N-tuple of markers Mn;
a step of determining, from a prediction model and the N-tuple of markers Mn relating to the subject, the predicted value of the K-tuple of marker Mk relating to the subject at time T+ΔT.

In one embodiment, the method according to a second aspect of the invention comprises, after the step of determining the predicted value of the K-tuple of markers Mk at time T+ΔT:
a step of acquiring the K-tuple of markers Mk at time T+ΔT;
a step of modifying the prediction model.

In one embodiment, the duration ΔT is greater than or equal to 6 months.

In one embodiment, the duration ΔT is less than or equal to 60 months.

A third aspect of the invention relates to a device comprising means for implementing a method according to a first or second aspect of the invention.

A fourth aspect of the invention relates to a computer program comprising instructions that cause the device according to a third aspect of the invention to perform the steps of the method according to a first or second aspect of the invention.

A fifth aspect of the invention relates to a computer-readable medium on which the computer program according to a fourth aspect of the invention is recorded.

The invention and its various applications will be better understood upon reading the following description and upon examining the accompanying figures.

### BRIEF DESCRIPTION OF THE FIGURES

The figures are set forth by way of indicating and in no way limiting purposes for the invention.

FIG. 1 shows a flow chart of a method according to a first aspect of the invention.

FIG. 2 shows a schematic representation of a prediction model according to the invention

FIG. 3 shows a flow chart of a method according to a second aspect of the invention.

FIG. 4 shows a schematic representation of a device according to a third aspect of the invention.

### DETAILED DESCRIPTION OF AT LEAST ONE EMBODIMENT OF THE INVENTION

Unless otherwise specified, a same element appearing in different figures has a unique reference.

### Possible Markers

A marker may be selected from a brain imaging marker (especially an anatomical imaging marker or a functional imaging marker), a subject cognitive score, a subject motor score, a subject autonomy score and a subject mood score.

A brain imaging marker may comprise an imaging marker indicative of the volumetry of at least one part of the brain or spinal cord (corresponding to an anatomical imaging marker), which may be derived from a nuclear magnetic resonance (MRI) image of at least one part of the brain or spinal cord. These markers may especially relate to the volumetry of a part of the subject's brain selected from hippocampal volume, whole brain volume, cerebellum volume, volume of subcortical structures, cortical thickness and/or opening of cortical-cerebral sulci. The brain imaging marker may also include a marker relating to lesion load, such as the volume of white matter lesions.

A brain imaging marker may include a functional imaging marker. Functional imaging parameters are determined by positron emission tomography (PET) or single photon emission computed tomography (SPECT). The latter allow the measurement of metabolic or molecular activity by virtue of the injection of a radioactive product, thus revealing certain biological processes, depending on the tracer used. The functional imaging marker may therefore include a marker relating to glucose metabolism, a marker relating to amyloid load and/or a marker relating to the dopaminergic system. For example, glucose metabolism (assessed by measuring glucose levels in different zones of the brain) may be determined by ¹⁸F-FDG PET, amyloid load (corresponding to the level of amyloid plaques) by amyloid PET, or the dopaminergic system (corresponding to the level of dopamine in the striatum and the general state of the dopamine transport system) may be determined by ¹²³I-FP-CIT SPECT (DaTscan). A functional imaging marker may include a marker for the measurement of the Blood Oxygen Level Dependent (BOLD) signal. This is the measurement of the variation in the amount of oxygen carried by haemoglobin: this change is related to neuronal activity in the brain, and is measured with functional magnetic resonance imaging (fMRI) techniques.

A marker can be related to the severity of a stroke and/or its possible after-effects on the patient, such as the volume of the infarcted zone. These markers are derived from an MRI of at least one part of the brain. The marker may comprise a marker indicative of white matter integrity, such as a measure of mean water diffusivity in a given brain region, for example measured with diffusion-weighted imaging (DWI) techniques.

A marker may be relative to the presence of some genes, such as the APOE gene, for example measured by a study of the subject's DNA from blood or saliva tests.

A marker may also relate to demographic data of the subject. By demographic data, it is meant in particular data selected from the socio-cultural level, sex and/or age of the subject. The marker may also consist of a score in the Socio-Economic Status Scale (SESS).

A marker may also be related to a cognitive score. A cognitive score is understood to be a parameter defining a subject's ability to remember and process information, whether visual or verbal, especially executive and instrumental functions measuring attention, planning and language use. The latter can be measured by various methods known to the skilled person. For example, it may be a cognitive test chosen from among the MMSE (Mini-Mental State Examination, or Folstein Test), ADAS-Cog (Alzheimer Disease Assessment Scale—Cognitive), 6-CIT (Six Item Cognitive Impairment Test) or GPCOG (General Practitioner assessment of Cognition) tests. Other tests that can be used, for example, are MOCA (Montreal Cognitive Assessment), BEC 96 (Cognitive Assessment Battery) or the Mattis scale. For example, the MMSE test provides an overall assessment of a person's cognitive state. It assesses orientation, learning, attention, calculation and language skills. The score obtained by this test can be used in the scope of the invention.

A marker can be related to a motor score. A motor score is a score representing an examination of motor functions such as walking, balance, the ability of a muscle to exert force against resistance. This may be measured by different methods, and may be, for example, a test selected from the Movement Disorder Society's revision of the Unified Parkinson Disease Rating Scale (MDS-UPDRS), the Expanded Disability Status Scale (EDSS) or Berg Balance Scale (BBS).

A marker can be related to an autonomy score. An autonomy score is understood to be the level of dependence and loss of autonomy of the subject, such as the level of autonomy for personal hygiene, locomotion or the management of personal finances. The latter can be measured by various methods, for example by a test chosen from IADL (Instrumental Activities of Daily Living) or FAQ (Functional Activities Questionnaire).

A marker can be related to a mood score. A mood score is understood to be a score that represents variations in the patient's moods, such as the level of severity of the patient's depressive or anxiety state. The latter may be measured by different methods, and may be, for example, a test selected from Depressive Mood Scale (DHS), Depression Anxiety Stress Scales (DASS) or Beck Depression Inventory (BDI).

Such markers may be representative of states preceding Alzheimer's disease, vascular dementia, dementia with Lewy bodies, frontotemporal lobar degeneration, Parkinson's disease, Huntington's disease, multiple sclerosis, amyotrophic lateral sclerosis, stroke, epilepsy, bipolar disorder, schizophrenia, autism, depression, post-traumatic disorders or head trauma.

### Method for Determining a Prediction Model

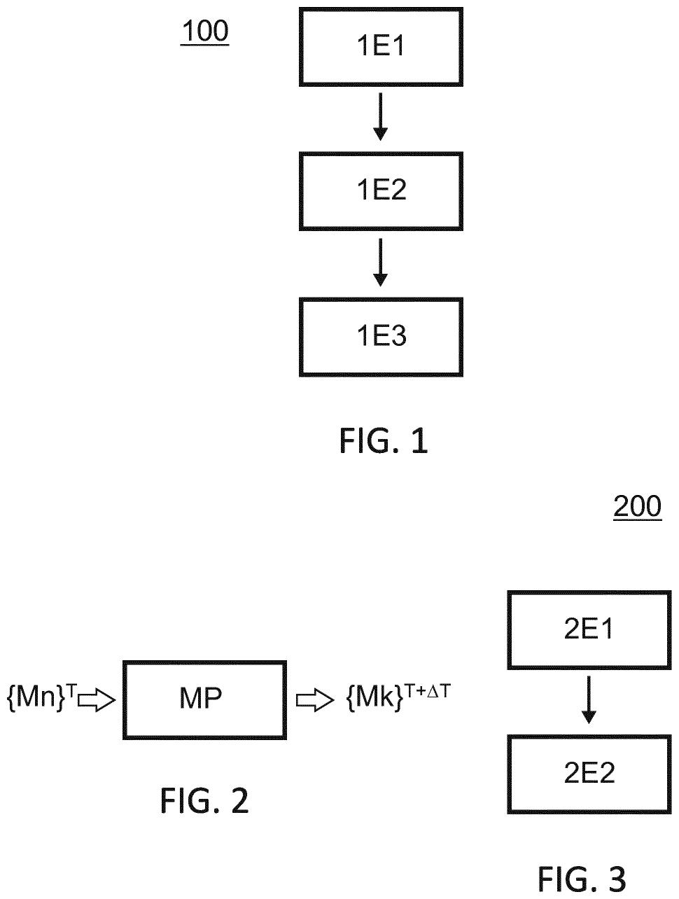

A first aspect of the invention illustrated in FIG. 1 and FIG. 2 relates to a method 100 for determining a prediction model MP, from an N-tuple of markers Mn, of the value of a K-tuple of markers Mk for aiding in the prognosis of pathologies of the central nervous system. In one embodiment, K is between 1 (inclusive) and 20 (inclusive), that is the K-tuple of markers Mk comprises a number of markers between 1 (inclusive) and 20 (inclusive). In one embodiment, K=1 (i.e. the K-tuple comprises only one marker Mk).

The method 100 according to a first aspect of the invention comprises, for each subject of a plurality of subjects, a step 1E1 of acquiring at a time T0 an N-tuple of markers Mn so as to obtain a plurality of N-tuple of markers Mn. This acquisition step may be carried out by using one or more images, by an operator entering the markers and/or by retrieving said markers from a database. In one embodiment, the number of subjects is greater than or equal to 100 (one hundred). It will be understood here that the markers Mn are identical from one subject to another, only the value of said markers may be different from one subject to another. Thus, each subject can be characterised by an N-tuple, said N-tuple being comprised of N markers Mn. For example, the N-tuple may comprise a functional imaging marker of the subject, a cognitive marker of the subject, an autonomy marker of the subject, a motor score marker (or motor marker) of the subject and/or a mood score marker (or mood marker) of the subject, the value of these different markers being generally different from one subject to another.

The method 100 then comprises, for each subject of the plurality of subjects, a step 1E2 of acquiring at a time T* greater than or equal to T0 a K-tuple of markers Mk so as to obtain a plurality of K-tuples of markers Mk. This acquisition step may be carried out by using one or more images, by an operator entering the markers and/or by retrieving said markers from a database. In the same way as above, it will be understood here that the markers Mk are identical from one subject to another, only the value of said markers may be different from one subject to another. For example, the K-tuple may include a functional imaging marker of the subject, a cognitive marker of the subject, an autonomy marker of the subject, a motor marker of the subject and/or a mood marker of the subject, the value of these different markers being generally different from one subject to another.

The method 100 finally comprises a step 1E3 of determining, from the plurality of N-tuples of markers Mn and the plurality of K-tuples of markers Mk, a prediction model MP making it possible to associate any N-tuple of markers Mn acquired at a time T with a K-tuple of markers Mk at a time T+ΔT with ΔT=T*−T0.

A model for predicting the value of a K-tuple of markers Mk from an N-tuple of markers Mn it is therefore understood to be a model which, taking an N-tuple of markers Mn as input corresponding to a subject and measured at a time T, makes it possible to establish the value of the K-tuple of markers Mk at a time T+ΔT with ΔT=T*−T0 for the subject in question. This prediction is made possible by the fact that the inventors have found that it is possible to reliably predict the course of variable parameters and in particular imaging phenotypes of a subject (here the K-tuple of markers Mk), by taking a limited number of relevant parameters (here the N-tuple of Markers Mn) into account, routinely determined during examinations following a consultation for disorders that may be related to an impairment of the central nervous system. In other words, although these parameters change over time in different ways depending on the subject and the type of damage, the speed and type of course of a marker (here the K-tuple of markers Mk) can be predicted by taking the value of a set of variable relevant markers (here the N-tuple of Markers Mn) into account. The prediction of the value of a K-tuple of markers Mk at the end of a given period of time constitutes a tool to assist in the management of patients; the value of these markers Mk, associated with clinical observations, is one of the steps allowing the clinician to predict the course of symptoms and more generally to better define the type of pathology a patient has.

In one embodiment, at least one of the markers of the N-tuple of markers Mn or the K-tuple of markers Mk is an imaging marker or a biological marker. Also, in one exemplary embodiment, the method comprises for each subject of the plurality of subjects, a step of performing one or more imaging procedures (e.g. magnetic resonance imaging of at least one part of the brain or spinal cord). The method then comprises, for each imaging procedure thus performed, a step of calculating, for each subject, at least one marker (e.g. a marker indicative of the volumetry of a part of the brain or spinal cord). Preferably, the N-tuple of markers Mn or the K-tuple of markers Mk comprises at least one anatomical imaging marker derived from an image acquired by MR.

In one embodiment, T*=T0, i.e. knowledge of the marker N-tuple Mn of a subject at time T0 enables the value of the K-tuple of markers Mk to be predicted at the same time.

In one embodiment, the step of acquiring an N-tuple of markers Mn so as to obtain a plurality of N-tuples of markers Mn is performed for a plurality of times Tᵢ (with i a natural number), the step of determining a prediction model taking the N-tuple of markers Mn into account for each time Tᵢ of the plurality of times Tᵢ. Indeed, the markers can change over time in a linear, logarithmic or exponential manner. The resulting prediction model MP will therefore be different for each course profile. The use of a plurality of times Tᵢ for the determination of a prediction model MP will, in the case of non-trivial (e.g. non-linear) courses, allow to obtain a more accurate prediction model. In the case of a plurality of times Tᵢ, the time between time Tᵢ and time Tᵢ+1 will be noted as ΔTᵢ. Moreover, the definition of ΔT is kept, i.e. ΔT=T*−T0 (with T0 equal to Tᵢ for i=0).

In one embodiment, the N-tuple of markers Mn comprises at least one marker Mn, preferably at least 2 markers Mn. In other words, N is greater than or equal to 1 or even greater than or equal to 2. However, a greater number of Markers Mn can be contemplated, such as a number of Markers Mn between 1 and 50 (i.e. N is between 1 and 50), or even between 2 and 10 (i.e. N is between 2 and 10).

In one embodiment, the N-tuple of markers Mn comprises at least one imaging marker indicative of the volumetry of a portion of the subject's brain selected from hippocampal volume, whole brain volume, cerebellum volume, volume of subcortical structures, cortical thickness and opening of cortical-cerebral sulci, said marker being derived from a magnetic resonance image of at least one part of the subject's brain.

In one embodiment, the N-tuple of markers Mn comprises at least one imaging marker indicative of lesion load, such as white matter lesion volume, said marker being derived from a magnetic resonance image of at least one part of the subject's brain or spinal cord.

In one embodiment, the N-tuple of markers Mn comprises at least one brain functional imaging marker selected from markers indicative of glucose metabolism, markers indicative of amyloid load, markers indicative of the dopaminergic system, and markers indicative of the level of brain oxygenation.

In one embodiment, the N-tuple of markers Mn comprises at least one marker selected from a cognitive marker of the subject; a motor marker of the subject; a mood marker of the subject; a demographic marker of the subject; a marker of the subject's autonomy; and/or a marker relating to the stage of progress of the subject in the disease.

In one embodiment, the N-tuple of markers Mn comprises at least one marker indicative of the concentration in the cerebrospinal fluid of at least one protein selected from Tau, P-tau and Abeta42 proteins, the measurement of said concentration being performed in vitro.

In one embodiment, the N-tuple of markers Mn comprises at least one marker relating to the mode of pharmaceutical molecule taken by the subject, that is the drug treatment taken by the subject and the dosage of this treatment.

In one embodiment, the N-tuple of markers Mn comprises at least one marker relating to the location and/or number of white matter lesions in the brain or spinal cord.

In one embodiment, the K-tuple of markers Mk comprises at least one imaging marker indicative of the volumetry of a part of the subject's brain selected from hippocampal volume, whole brain volume, cerebellum volume, volume of subcortical structures, cortical thickness, and opening of cortical-cerebral sulci, said marker being derived from a magnetic resonance image of at least one part of the subject's brain.

In one embodiment, the K-tuple of markers Mk comprises at least one imaging marker indicative of lesion load, such as white matter lesion volume, said marker being derived from a magnetic resonance image of at least one part of the brain or spinal cord of the subject.

In one embodiment, the K-tuple of markers Mk comprises at least one brain functional imaging marker selected from markers indicative of glucose metabolism, markers indicative of amyloid load, markers indicative of the dopaminergic system, and markers indicative of level of brain oxygenation.

In one embodiment, the K-tuple of markers Mk comprises at least one cognitive marker of the subject; a motor marker of the subject; a mood marker of the subject; an autonomy marker of the subject; and/or a marker relating to the progress of the subject in the disease.

### Management of Outliers or Missing Data

During the step of acquiring at a time T0 an N-tuple of markers Mn or the step of acquiring at a time T* greater than or equal to T0 a K-tuple of markers Mk, it is possible that acquisition errors lead to outlier data which result in outlier or missing markers.

Such data (or markers) can lead to a degradation of the prediction model. It is therefore important to identify and correct them.

For this end, in one embodiment, the method comprises, after the step of acquiring at a time T0 an N-tuple of markers Mn, preferably after the step of acquiring at a time T* greater than or equal to T0 a K-tuple of markers Mk, and before the step of determining a prediction model, a step of correcting the marker outliers.

In one embodiment, the step of correcting marker outliers comprises, for each marker Mn of the N-tuple of markers Mn, a substep of determining the number of subjects for which the value of said marker Mn is judged to be an outlier. At the end of this sub-step, for each marker Mn of the N-tuple, the number of subjects for which the value of said marker is considered to be an outlier is available.

The step of correcting marker outliers then comprises, for each marker Mn, if the number of subjects for which the value of the marker Mn in question is considered to be an outlier is greater than a threshold number of subjects, a sub-step of deleting the marker Mn in question, said marker not being taken into account during the step of determining a prediction model. In one embodiment, the threshold number of subjects is equal to 5% (five percent) of the total number of subjects of the plurality of subjects.

The step of correcting marker outliers then comprises for each marker Mn, if the number of subjects for which the value of the marker Mn in question is judged to be an outlier is less than or equal to the threshold number of subjects, a substep of replacing, for the subjects concerned, the value of the marker Mn in question by the value of the closest quartile of said marker Mn.

The value Xi of a marker Mn is judged to be an outlier for a subject if it does not satisfy the following relationship:

Med−3σ_{b}≤Xᵢ≤Med+3σ_{b}

where Med is the median of the values Xᵢ of the marker Mn for the plurality of subjects, σ_{b} the value of a deviation such that σ_{b}=b×DMed with b a predefined coefficient and DMed=median(|X₁−Med|). It will be understood here that the function median(x₁, . . . , xₙ) corresponds to the median value of the values x₁, . . . , xₙ. The median is preferred here to the mean, as it is more robust to the presence of outliers. In one embodiment, the value of the factor b may depend on the type of distribution governing the values of the marker in question. For example, if the marker values obey a normal distribution, then the value of b may be chosen such that b=1.4826. In the same example, if the values do not obey a normal distribution, then the value of b can be chosen such that b=1/Q(0.75), with Q(0.75) 0.75 quantile of that distribution. For more detail, it is possible to refer to the article Leys, C., Ley, C., Klein, O., Bernard, P., & Licata, L. (2013). Detecting outliers: Do not use standard deviation around the mean, use absolute deviation around the median. Journal of Experimental Social Psychology, 49(4), 764-766.

In the same way, it can happen that for some subjects, no value is associated with a given marker. In this case, it is necessary to correct this absence.

To this end, in one embodiment, the method comprises, after the step of acquiring at a time T0 an N-tuple of markers Mn, preferably after the step of acquiring at a time T* greater than or equal to T0 a K-tuple of markers Mk, and before the step of determining a prediction model, a step of correcting the missing marker values.

When the number of missing markers for a given subject is too large, it may be detrimental to take this subject into account when determining the prediction model MP. In order to take this aspect into account, in one embodiment, the step of correcting the missing marker values comprises, for each subject of the plurality of subjects, when the number of missing markers Mn for said subject is greater than a threshold number of markers, a substep of removing the subject from the plurality of subjects, said subject not being taken into account during the step of determining a prediction model MP. In one embodiment, for markers Mn, the threshold number of markers is equal to 5% (five percent) of the number of markers in the N-tuple of markers Mn.

In one embodiment, the added value for a given marker Mn is equal to the mean of said marker for the three closest subjects. For this, the step of adding a predetermined value for missing values of markers Mn comprises a substep of determining the three closest subjects to the subject associated with a missing marker Mn value, this proximity determination being based on the values of non-missing markers Mn in said subject; a substep of calculating the mean value of the missing marker for the three closest subjects; and a step of adding the value of the missing marker, said value being equal to the mean value calculated in the substep of calculating the mean value of the missing marker.

As a remark, no correction of missing data is performed on the markers Mk of the K-tuple of markers Mk. Indeed, a subject is taken into account in the determination of the prediction model MP if, and only if, the K-tuple of markers Mk associated therewith does not include any missing marker Mk.

Moreover, when the step of acquiring an N-tuple of markers Mn so as to obtain a plurality of N-tuple of markers Mn is performed for a plurality of times Ti, the correction of the outlier or missing markers Mn is performed on the plurality of N-tuple of markers Mn obtained for each of the times Ti.

### Quality Control of the Imaging Data

The markers of the N-tuple of markers Mn or K-tuple of markers Mk may include medical imaging markers. It can therefore be advantageous to make sure of the good quality of said images.

For this, a method according to the invention comprises, after the step of acquiring at a time T0 an N-tuple of markers Mn, preferably after the step of acquiring at a time T* greater than or equal to T0 a K-tuple of markers Mk, and before the step of determining a prediction model, a step of controlling the quality of the imaging data. Preferably, this control step is implemented before the outlier correction step if the method includes such a step.

In particular, the control step comprises a first sub-step of checking compliance with the acquisition procedure. This verification can be carried out by comparing the values of the acquisition parameters with recommended values to ensure the proper operation of the subsequent analyses. These acquisition parameter values are for example obtained from the DICOM files. The recommended values are empirically determined values and depend on the type of magnetic resonance imaging used for the acquisition, the imaging markers to be extracted from the acquisition and the methods used to extract these markers. For example, an anatomical imaging marker will preferably be extracted by segmentation of a 3DT1 type acquisition with, among other parameters, a spatial resolution of 256×256×192 mm³ and a voxel size of 1×1×1 mm³. At the end of this sub-step, the imaging markers associated with an acquisition that does not comply with the procedure are considered as missing. They could then be processed as described above in the context of outlier or missing data management.

The control step also includes, when the image has been obtained in compliance with the procedure, a step for verifying the quality of the images that have allowed the determination of the value of the marker, that is the possibility or not of obtaining reliable analysis results from said images. This can be assessed, for example, using automated tools for calculating measures derived from the intensities of the images, measures that we will be called “descriptors”. These measures are classic in the field, for example these are a measure of SNR (Signal to Noise Ratio). At the end of this sub-step, each image is associated with a plurality of descriptors. The monitoring step then includes a sub-step of assessing the quality of each plurality of descriptors using a classifier, for example a Support Vector Machine (SVM) classifier, which compares the value of the plurality of descriptors with those of a training base of subjects. A training base is set up for the design of the classifier, with several hundred patients. Analysis results of the imaging data are available for each patient. In addition, for each of the subjects in the training base, the reliability of the different results obtained after analysis of these data is visually assessed. This assessment will constitute the “gold standard”. Thus, the classifier makes it possible to automatically assess the expected reliability of the results of the analysis of imaging data. For example, one or more combinations of values corresponding to a quality “the analysis results will be reliable”, one or more other combinations corresponding to a quality “the analysis algorithms will not work at all on these data” and one or more others corresponding to a quality “the analysis algorithms will work, but the results will not be reliable” are associated. In general, the training base is built up using at least 200 subjects. For more detail, the following articles can be referred to: Pizarro, R. A., Cheng, X., Barnett, A., Lemaitre, H., Verchinski, B. A., Goldman, A. L., . . . & Weinberger, D. R. (2016). Automated quality assessment of structural magnetic resonance brain images based on a supervised machine learning algorithm. Frontiers in neuroinformatics, 10, 52; and Esteban, O., Birman, D., Schaer, M., Koyejo, O. O., Poldrack, R. A., & Gorgolewski, K. J. (2017). MRIQC: Advancing the automatic prediction of image quality in MRI from unseen sites. PloS one, 12(9), e0184661.

It is generally preferable to test the classifier before implementing it.

This is done by means of a so-called test base (usually made up of about 100 subjects), for which a visual assessment of the reliability of the analysis results is also available. This validated classifier is then used to determine the quality level of a new image.

When imaging data are deemed reliable, tools conventionally used by the skilled person are used to automatically segment brain structures and extract markers such as hippocampal volume (normalised to intracranial volume). Examples of such tools are SPM (www.fil.ion.ucl.ac.uk/spm), FSL (http://fsl.fmrib.ox.ac.uk/fsl/fslwiki/) or Freesurfer (http://freesurfer.net) software.

Finally, it may be useful to check the quality of the image segmentation. For this, the quality of the segmentations is assessed using automated tools, measuring for example the intensities in the segmentation masks and in their vicinity. The extracted intensities are fed into a classifier, for example an SVM classifier, in order to assess the quality of the segmentations. Verification is then carried out in the same way as above.

When the quality of the plurality of descriptors is above a predetermined threshold, the imaging marker is retained. However, if the quality of the plurality of descriptors is below a predetermined threshold, the imaging marker is considered as missing. It can then be treated as described above within the scope of managing outliers or missing data.

### Method for Predicting the Value of a K-Tuple of Markers Mk

Once a prediction model MP has been established using a method 100 according to a first aspect of the invention, it is possible to use said prediction model MP to determine the future value of a marker or several markers. For this, a second aspect of the invention illustrated in FIG. 3 relates to a method 200 for predicting the course of a K-tuple of markers Mk for aiding in the prognosis of pathologies of the central nervous system using a prediction model MP obtained by implementing a method 100 according to a first aspect of the invention.

The method 200 according to a second aspect of the invention comprises a step 2E1 of acquiring at a time T an N-tuple of markers Mn relating to a subject. This acquisition step may be carried out by using one or more imaging procedures, by an operator entering the markers and/or by retrieving said markers from a database. It will be understood here that the acquired N-tuple comprises the same markers Mn as the N-tuple that made it possible to determine the prediction model MP upon implementing a method 100 according to a first aspect of the invention.

The method 200 also comprises a step 2E2 of determining, from a prediction model and the N-tuple of markers Mn relating to the subject, the predicted value of the K-tuple of markers Mk relating to the subject at time T+ΔT. Preferably, the duration ΔT is greater than or equal to 6 months. Preferably, the duration ΔT is less than or equal to 60 months.

In one embodiment, the method according to a second aspect of the invention comprises, after the step 2E2 of determining the predicted value of the K-tuple of markers Mk at time T+ΔT, a step of acquiring the K-tuple of markers Mk at time T+ΔT; and a step of modifying the prediction model. This acquisition step may be carried out by using one or more imaging procedures, by an operator entering the markers and/or by retrieving said markers from a database. In this way, the process 200 allows the prediction model to be continuously refined and improved by virtue of the new measurements made in the normal context of the prediction process. In other words, it is possible to change the prediction process 200 over time by changing the prediction model MP over time, to take the parameters recorded during its implementation into account.

The prediction method 200 according to a second aspect of the invention is in particular adapted to predict the course of a marker Mk of a neurological state of a subject suffering from cognitive disorders or of a subject suffering from motor disorders related to an impairment of the central nervous system. In one exemplary embodiment, the N-tuple of markers Mn comprises a marker of whole brain volume, a marker of basal ganglia volume and a motor marker measured using an EDSS method. The K-tuple of marker in turn includes a motor marker as measured by an EDSS method.

The prediction method 200 according to a second aspect of the invention is particularly adapted for predicting the course of a marker of hippocampal volume. In this exemplary embodiment, the N-tuple of markers Mn comprises a marker of hippocampal volume, a marker of whole brain volume and a marker representative of the MMSE score. In addition, the K-tuple of markers Mk comprises a marker of hippocampal volume.

The prediction method 200 according to a second aspect of the invention is particularly adapted to predict the course of a marker representative of the MMSE score with ΔT=24 months. In this exemplary embodiment, the N-tuple of markers Mn comprises a marker related to gender, age, a marker of education level, a marker representative of the MMSE score, a marker of white matter volume, a marker of grey matter volume, a marker of hippocampal volume and a marker of amygdala volume. In addition, the K-tuple of markers Mk includes a marker representative of the MMSE score. The prediction model used is then of the “ridge” type.

The prediction method 200 according to a second aspect of the invention is particularly adapted to predict the course of a marker representative of the amyloid load with ΔT=0. In this exemplary embodiment, the N-tuple of markers Mn comprises sex, age, a marker of education level, a marker representative of genetic status (APOE4), a marker representative of MMSE score, a marker of white matter volume, a marker of grey matter volume, a marker of hippocampal volume and a marker of amygdala volume. In addition, the K-tuple of markers Mk comprises a marker representing amyloid load. The prediction model used is then of the “logistic regression” type.

Similarly, a prediction method 200 according to a second aspect of the invention is particularly adapted to predict the course of a marker Mk of a neurological state. For this, in one embodiment, the N-tuple comprises a marker of whole brain volume, a marker of hippocampal volume and a cognitive marker obtained using an ADAS method. In addition, the K-tuple of markers Mk includes an amyloid marker obtained by a PET imaging technique.

For example, for multiple sclerosis, lesion reading alone is not a sufficient prognostic marker. The method 200 according to the invention will help in the modulation of treatments and the tailored management of patients, with the automated measurement of markers representative of overall atrophy, volume of basal ganglia, cerebellum, spinal cord, for example, combined with clinical data, such as markers representative of the level of disability (e.g. EDSS) or markers representative of measurements of cognitive function (SDMT—Symbol Digit Modalities Test, which is a test that asks the subject to substitute numbers and symbols in 90 seconds, for example). Thus, the N-tuple of markers Mn may comprise all or some of these markers.

In one embodiment, when the step of acquiring a N-tuple of markers Mn so as to obtain a plurality of N-tuple of markers Mn used for determining the prediction model is performed for a plurality of times Ti, the step 2E1 of acquiring the method according to a second aspect of the invention is performed for a plurality of times Tj. Moreover, the time separating two successive acquisitions noted ΔTⱼ (and equal to Tⱼ₊₁−Tⱼ) is equal to ΔTi (as defined previously).

### Associated Device

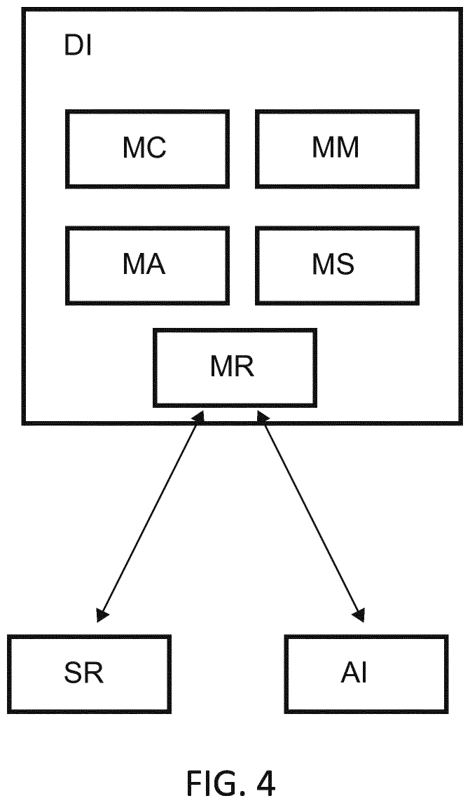

A third aspect of the invention illustrated in FIG. 4 relates to a device DI comprising means for implementing a method 100, 200 according to a first or second aspect of the invention. In one exemplary embodiment, the 30 device comprises a computing means MC (e.g. a processor) and a memory MM (for example a RAM memory) associated with said computing means MC. The memory MM is configured to store instructions as well as data necessary for the implementation of a method 100, 200 according to a first or second aspect of the invention. In one exemplary embodiment, the device DI also comprises input means MS and display means MA (for example a keyboard, a screen, a touch screen, etc.) in order especially to allow one or more operators to input all or part of the markers necessary for the implementation of a method 100, 200 according to a first or a second aspect of the invention. In one embodiment, the device DI also comprises connection means MR (for example a network card) in order to be able to exchange with a server SR, said server SR storing all or part of the markers necessary for the implementation of a method 100, 200 according to a first or a second aspect of the invention. In one exemplary embodiment, the device DI also comprises connection means MR (e.g. a network card) in order to be able to exchange with one or more imaging devices AI so as to trigger one or more imaging procedures and/or retrieve all or part of the data allowing the generation of the markers necessary for the implementation of a method 100, 200 according to a first or second aspect of the invention.

1. A method for determining a prediction model, from an N-tuple of markers Mn, of the value of a K-tuple of markers Mk for aiding in a prognosis of pathologies of a central nervous system, the method comprising:
for each subject of a plurality of subjects, a step of acquiring at a time T0 an N-tuple of markers Mn so as to obtain a plurality of N-tuples of markers Mn;
for each subject of the plurality of subjects, a step of acquiring at a time T* greater than or equal to T0 a K-tuple of markers Mk so as to obtain a plurality of K-tuples of markers Mk;
a step of determining, from the plurality of N-tuples of markers Mn and the plurality of K-tuples of markers Mk, a prediction model making it possible to associate any N-tuple of markers Mn acquired at a time T with a K-tuple of markers Mk at a time T+ΔT with ΔT=T*−T0.

2. The method according to claim 1, wherein at least one of the markers of the N-tuple of Markers Mn or the K-tuple of markers Mk is an imaging marker or a biological marker.

3. The method according to claim 1, wherein at least one of the markers of the N-tuple of markers Mn is selected from:
an imaging marker indicative of the volumetry of a part of the subject's brain selected from hippocampal volume, whole brain volume, cerebellum volume, volume of subcortical structures, cortical thickness and opening of cortical-cerebral sulci, said marker being derived from a magnetic resonance image of at least one part of the subject's brain;
an imaging marker indicative of lesion load, said marker being derived from a magnetic resonance image of at least one part of the subject's brain or spinal cord; and
a brain functional imaging marker selected from markers indicative of glucose metabolism, markers indicative of amyloid load, markers indicative of the dopaminergic system and markers indicative of the level of brain oxygenation.

4. The method according to claim 1, wherein at least one of the markers of the N-tuple of markers Mn is selected from:
a cognitive marker of the subject
a motor marker of the subject
a mood marker of the subject;
a demographic marker of the subject;
a marker of the subject's autonomy;
a marker relating to the subject's stage of progress in the disease.

5. The method according to claim 1, wherein at least one of the markers of the K-tuple of markers Mk is selected from:
an imaging marker indicative of the volumetry of a part of the subject's brain selected from hippocampal volume, whole brain volume, cerebellum volume, volume of subcortical structures, cortical thickness and opening of cortical-cerebral sulci, said marker being derived from a magnetic resonance image of at least one part of the subject's brain;
an imaging marker indicative of lesion load, said marker being derived from a magnetic resonance image of at least one part of the subject's brain or spinal cord; and
a brain functional imaging marker selected from markers indicative of glucose metabolism, markers indicative of amyloid load, markers indicative of the dopaminergic system and markers indicative of the level of brain oxygenation.

6. The method according to claim 1, wherein at least one of the markers of the K-tuple of markers Mk is selected from:
a cognitive marker of the subject;
a motor marker of the subject;
a mood marker of the subject;
a marker of the subject's autonomy;
a marker of the subject's stage of progress in the disease.

7. The method according to claim 1, further comprising, after the step of acquiring at a time T0 an N-tuple of marker Mn and before the step of determining a prediction model, a step of correcting the marker outliers.

8. The method according to claim 1, further comprising, after the step of acquiring at a time T0 an N-tuple of marker Mn and before the step of determining a prediction model, a step of adding a predetermined value for the missing values of markers Mn.

9. The method according to claim 1, further comprising, after the step of acquiring an N-tuple of markers Mn at a time T0 and before the step of determining a prediction model, a step of controlling the quality of the imaging markers.

10. The method according to claim 1, wherein the step of acquiring an N-tuple of Markers Mn so as to obtain a plurality of N-tuples of Markers Mn is performed for a plurality of times T0, the step of determining a prediction model taking the N-tuples of markers Mn into account for each time T0 of the plurality of times T0.

11. A method for predicting a course of a K-tuple of markers Mk in a subject for aiding in a prognosis of pathologies of a central nervous system using a prediction model of a K-tuple of markers Mk obtained using a method according to claim 1, the method comprising:
a step of acquiring at a time Tan N-tuple of marker Mn;
a step of determining, from a prediction model and the N-tuple of markers Mn relating to the subject, the predicted value of the K-tuple of marker Mk relating to the subject at time T+ΔT.

12. The method according to claim 11, further comprising, after the step of determining the predicted value of the K-tuple of markers Mk at time T+ΔT:
a step of acquiring the K-tuple of markers Mk at time instant T+ΔT;
a step of modifying the prediction model.

13. A device comprising means for implementing a method according to claim 1.

14. A computer program comprising instructions which cause a device to perform the steps of the method according to claim 1.

15. A non-transitory computer-readable medium, on which the computer program according to claim 14 is recorded comprising machine executable instructions to perform the steps of the method according to claim 1.

16. The method according to claim 3, wherein the lesion load is a volume of white matter lesions.

17. The method according to claim 5, wherein the lesion load is a volume of white matter lesions.
